(19) United States
(12) Patent Application Publication
(10) Pub. No.: US 2008/0227182 A1
(43) Pub. Date: Sep. 18, 2008

(54) SYSTEMS AND METHODS FOR ENZYMATIC HYDROLYSIS OF LIGNOCELLULOSIC MATERIALS

(75) Inventors: Dwight E. Anderson, Puyallup, WA (US); Chundakkadu Krishna, Federal Way, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: WEYERHAEUSER COMPANY, Federal Way, WA (US)

(21) Appl. No.: 12/050,110

(22) Filed: Mar. 17, 2008

Related U.S. Application Data
(60) Provisional application No. 60/946,769, filed on Jun. 28, 2007.
(60) Provisional application No. 60/895,346, filed on Mar. 16, 2007.

(51) Int. Cl. C12M 1/00; C07G 17/00

(52) U.S. Cl. 435/267; 435/289.1; 435/294.1

(57) ABSTRACT

Systems and methods for enzymatic hydrolysis of lignocellulosic materials are provided. A system for enzymatic hydrolysis of lignocellulosic materials generally includes a reactor vessel configured to contain a mixture of lignocellulosic stock and enzymes. The reactor vessel includes a first agitator for mixing in the reactor vessel. The system further includes a recycle loop coupled to the reactor vessel for recycling the mixture from and returning to the reactor vessel and a second agitator for mixing in the recycle loop.

### CROSS-REFERENCES TO RELATED APPLICATIONS

This application claims the benefit of U.S. Provisional Application No. 60/895,346, filed Mar. 16, 2007, and U.S. Provisional Application No. 60/946,769, filed Jun. 28, 2007, the disclosures of which are hereby expressly incorporated by reference.

### BACKGROUND

Ethanol has widespread application as an industrial chemical, gasoline additive, or straight liquid fuel. It is the predominant alternative liquid fuel used in the United States, marketed as a 10% blend with gasoline where it provides value both as an octane enhancer and oxygenate. As a fuel or fuel additive, ethanol dramatically reduces air emissions while improving engine performance. It is also sold at up to 85% blend with gasoline and used in cars and trucks that have been manufactured or retrofitted to utilize this mixture. As a renewable fuel, ethanol reduces national dependence on finite and largely foreign fossil fuel sources while decreasing the net accumulation of carbon dioxide in the atmosphere. National concerns about dependence on imported petroleum, balance or payments, and global climate change have provided the impetus to find indigenous resources and technologies that can provide large quantities of alternative liquid fuels at reasonable costs. Ethanol produced from cellulosic biomass (cellulosic ethanol) is particularly attractive in this context.

Ethanol typically has been produced from sugars derived from feedstock high in starches or sugars, such as corn. Recovered paper, such as old corrugated containers (OCC), and other lignocellulosic materials have potential as substrates for ethanol production due to their availability, relatively high concentrations of cellulose, and low cost. In ethanol production from lignocellulosic materials, hydrolysis and fermentation steps are required. Hydrolysis can be carried out prior to or simultaneously with fermentation, either through acidic and/or enzymatic hydrolysis.

However, due to the high cost and the large amount of cellulose hydrolyzing enzyme required, successful utilization of lignocellulosic materials, particularly recovered paper, as a renewable carbon source has not yet been achieved. In enzymatic hydrolysis, mass transfer limitations generally limit the rate of hydrolysis of the lignocellulosic materials. For example, common industrial scale enzymatic hydrolysis processes using large agitated tanks can take at least 2-4 days to complete enzymatic hydrolysis as a result of the enzyme mass transfer limitations. Therefore, there exits a need to improve enzyme mass transfer in enzymatic hydrolysis processes of lignocellulosic materials for improved rates of hydrolysis to produce ethanol from recovered paper and other lignocellulosic materials at a lower cost and with improved efficiency.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A system for enzymatic hydrolysis of lignocellulosic materials is provided. The system generally includes a reactor vessel configured to contain a mixture of lignocellulosic stock and enzymes, the reactor vessel including a first agitator for mixing in the reactor vessel. The system further includes a recycle loop coupled to the reactor vessel for recycling the mixture from and returning to the reactor vessel, and a second agitator for mixing in the recycle loop.

A system for enzymatic hydrolysis of lignocellulosic materials is provided. The system generally includes a first reactor vessel configured to contain a mixture of lignocellulosic stock and enzymes, the first reactor vessel including a first agitator and a first vessel feed line, and a second agitator for promoting mixing in the first vessel feed line.

A method of enzymatic hydrolysis of lignocellulosic materials is provided. The method generally includes in a reactor vessel, agitating a mixture of lignocellulosic stock and enzymes. The method further includes recycling the mixture in a recycle loop extending from and returning to the reactor vessel, and further comminuting the mixture in the recycle loop for promoting effective enzyme mass transfer to and from lignocellulosic fibers in the lignocellulosic stock without enzyme deactivation.

A method of enzymatic hydrolysis of lignocellulosic materials is provided. The method generally includes in a first reactor vessel, agitating a mixture of lignocellulosic stock and enzymes, the first reactor vessel including a first agitator and a first vessel feed line. The method further includes further agitating the mixture in the first vessel feed line for promoting effective mass transfer of the enzymes to and from lignocellulosic fibers in the lignocellulosic stock without enzyme deactivation.

### DESCRIPTION OF THE DRAWINGS

The foregoing aspects and many of the attendant advantages of this disclosure will become more readily appreciated by reference to the following detailed description, when taken in conjunction with the accompanying drawings, wherein:

FIG. 1 is a process diagram of a method in accordance with one embodiment of the present disclosure for producing a hydrolysate and ethanol from lignocellulosic materials;

FIG. 2 is a process diagram of a method in accordance with another embodiment of the present disclosure for producing a hydrolysate and ethanol from lignocellulosic materials;

FIG. 3 is a graph showing a comparative amount of hexose sugars produced over time by hydrolysis of treated and untreated OCC hydrolyzed with 20 FPU of enzymes per gram of OCC, wherein the treated OCC was subjected to two-stage lignin deactivation with oxygen in an alkaline medium;

FIG. 4 is a graph comparing hexose and pentose sugar conversion theoretical percentage of OCC as a function of ten-minute Kappa number;

FIG. 5 is a graph comparing total sugar yield for different fiber types and lignin deactivation treatments;

FIG. 6 is a graph showing a comparative amount of hexose sugars produced over time by hydrolysis of untreated OCC hydrolyzed with 40 and 80 FPU of enzymes per gram OCC enzyme loading, wherein some of the samples were subjected to a refining process prior to hydrolysis;

FIG. 7 is a schematic of a system for enzymatic hydrolysis of lignocellulosic materials designed in accordance with one embodiment of the present disclosure;

FIG. 8 is a graph showing the proportion of lignocellulosic materials subjected to turbulence versus the intensity of the turbulence for the system of FIG. 1;

FIG. 9 is a graph showing total sugar conversion as a result of enzymatic hydrolysis versus time under various refining process conditions for OCC samples; and

FIG. 10 is a schematic of a system for enzymatic hydrolysis of lignocellulosic materials designed in accordance with another embodiment of the present disclosure.

### DETAILED DESCRIPTION

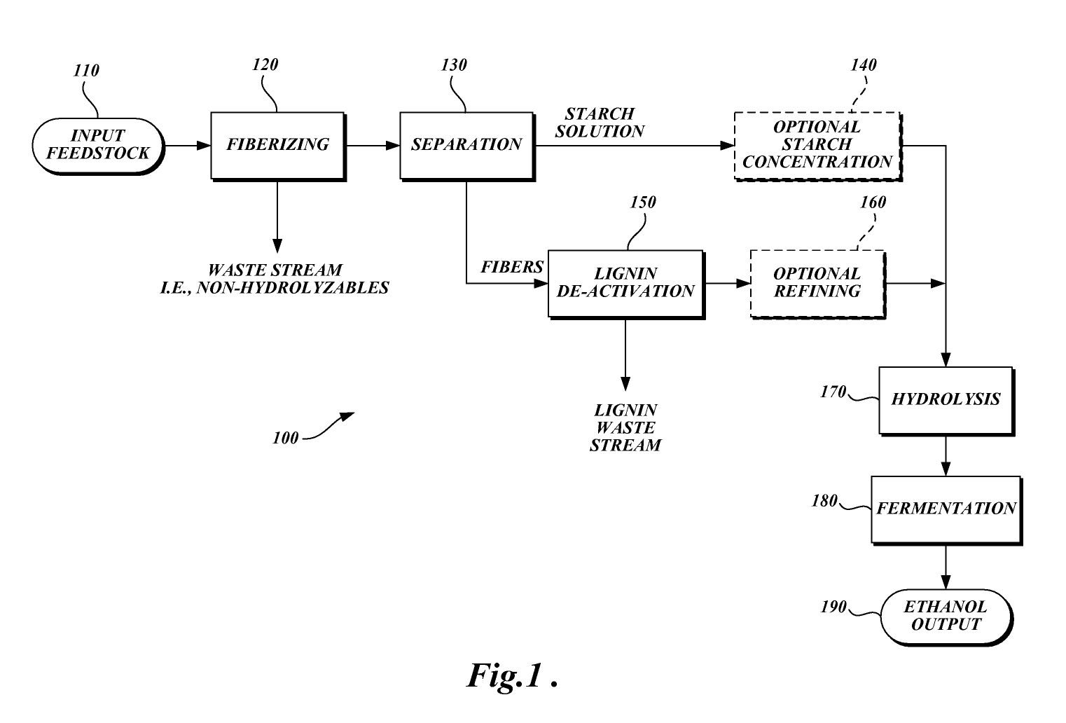

Embodiments of the present disclosure are generally directed to methods and compositions for producing a hydrolysate from lignocellulosic materials. Such a hydrolysate can be fermented to produce ethanol. Referring to FIG. 1, a process diagram 100 for a method of converting lignocellulosic materials to ethanol in accordance with one embodiment of the present disclosure is shown. In accordance with the illustrated embodiment of FIG. 1, the method includes fiberizing lignocellulosic materials, as represented by block 120, separating the lignocellulosic materials, as represented by block 130, deactivating the lignin in at least a portion of the lignocellulosic materials, as represented by block 150, and hydrolyzing the fiberized lignocellulosic materials with enzymes to produce a hydrolysate, as represented by block 170.

The method may further include fermenting the hydrolysate with a fermentation material, as represented by block 180, to produce ethanol, as represented by block 190. As will be described in greater detail below, the embodiments described herein are generally directed at minimizing the amount of hydrolyzing enzymes required by the methods to improve the yields of hydrolysis and fermentation and the feasibility of the methods from a cost perspective by reducing the chemical additives in the process.

As seen in the illustrated embodiment of FIG. 1, lignocellulosic feedstock 110 is collected and brought to the plant and, as mentioned above, the feedstock is fiberized, as represented by block 120. Various methods of fiberizing, including wet (chemical) and dry (mechanical) methods, are within the scope of the present disclosure. Suitable wet methods of fiberizing materials include hydropulping with water as well as other chemical cooks, including but not limited to the following processes: digestion of fibers, kraft pulping, steam explosion, soda cook, kraft cook, sulfate, sulfite, soda, and organosolve processes. Other suitable dry methods of fiberizing materials include hammermilling, fiberizing, and grinding. It should be appreciated that chemical and mechanical fiberizing processes may be suitably used alone or in combination.

During fiberization, non-hydrolysable contaminants, such as plastics, metals, and any undesirable components, may be separated from the feedstock as a waste stream of residual materials. It should be appreciated, however, that there may be additional residual material streams produced during other process steps of the method shown in FIG. 1. Representative residual materials include plastics from cleaning and fiberizing, lignin separated from cellulose during the lignin deactivation process, and residual yeast, lignin, and other non-fermented products from the fermentation process. It should further be appreciated that a combustible portion of the residual materials (for example, lignins) may be recycled and converted to energy to provide required heat for the operation of the methods described herein.

Suitable feedstock for use with the methods described herein includes lignocellulosic materials including but not limited to recovered paper (such as OCC, mixed paper, and newspaper), recycled wood materials, for example, from sawmill and urban wood waste, municipal solid waste, yard waste, and other non-virgin biomass, as well as virgin biomass including energy crops, wood materials, and other biomass high in cellulose and/or starch.

Suitable energy crops are regenerating lignocellulosic energy crops including but not limited to perennial plant species such as switch grass (including panicum virgatum and other varieties of the genus panicum), miscanthus (including miscanthus giganteus and other varieties of the genus miscanthus), giant reed (arundo donax), energy cane (saccharum spp.), and napier grass (pennisetum purpureum).

Recovered paper may include material that has been collected from end users and includes old corrugated containers (OCC), which include used containers and container plant cuttings. Recovered paper may also include, in various amounts, mixed paper, which includes paper of varied quality such as unsorted office papers, magazines, and unsorted household papers and old newspapers including unsold and household newspapers.

Lignocellulosic materials generally may include some or all of the following components: cellulose, hemicellulose, lignin, protein, and carbohydrates, such as starch and sugar.

As a non-limiting example of lignocellulosic properties, the physical and chemical properties of lignin depend on their plant source and processing conditions. In that regard, lignins can generally be grouped into three broad classes, including softwood or coniferous (gymnosperm), hardwood (dicotyledonous angiosperm), and grass or annual plant (monocotyledonous angiosperm) lignins. Grass and annual plants may have lignins having properties that are similar to hardwood lignins. Groundwood fibers (or mechanical pulp) may have lignin contents that are similar to either softwood or hardwood fibers depending on whether the source is softwood groundwood or hardwood groundwood. Moreover, recovered paper, which has already been processed once, may generally have a lower starting lignin content for the purpose of the processes described herein than a similar type of virgin biomass.

Certain types of lignins are harmful to the enzymatic hydrolysis process because they tend to bond with the enzymes used in enzymatic hydrolysis, causing the enzymes to be less effective during the hydrolysis process. The presence of lignin, particularly certain types of lignin, therefore decreases the efficiency of the enzymatic hydrolysis process and increases the costs associated with enzymatic hydrolysis by requiring additional enzymes. It is thus advantageous to remove and/or modify at least a portion of the lignin prior to enzymatic hydrolysis. In accordance with embodiments of the present disclosure, lignin can be partially or wholly removed from the lignocellulosic materials and/or modified by a lignin deactivation process, which will be described in greater detail below.

Separation of the lignocellulosic materials will now be described in greater detail. Because sources of lignocellulosic materials may include a mixture of components, it may further be advantageous to separate lignocellulosic materials prior to the lignin deactivation process so that appropriate lignin deactivation processes can be performed for different materials. For example, a portion of the feedstock may require lignin deactivation, while another portion may not. Alternatively, a portion of the feedstock may be subjected to a specific lignin deactivation process, while another portion may be subjected to a different lignin deactivation process. Different portions of the lignocellulosic materials may include but are not limited to starch, softwood fibers, hardwood, fibers, mechanical pulp fibers, grass and annual plant fibers, and any combination thereof.

As a non-limiting example, OCC is a feedstock that may be suitably separated into its components before lignin deactivation processing due to the presence of numerous components including hardwood, softwood, and starch. In that regard, in a typical corrugated cardboard construction, the outer liners may be predominately made from softwood fibers, while the corrugated medium may be predominately made from hardwood fibers. In addition, 0.5-10% starch is typically added to a corrugated cardboard construction. Such a construction may result in OCC that contains greater than about 60% of cellulose, about 10-18% of lignin, about 10-18% of hemicellulose, and about 0.5-10% starch and modified starches by weight. Because the cellulose makeup of OCC generally contains a mix of hardwood and softwood fibers, the lignin present in the OCC feedstock stream will be varied and will therefore require different lignin deactivation processes. Moreover, the starch component does not need to undergo a lignin deactivation process because there is little to no lignin content in starch.

In order to minimize processing and chemical additives during the lignin deactivation process, a separation of lignocellulosic materials is within the scope of the present disclosure. As represented by block 130 in the illustrated embodiment of FIG. 1, the lignocellulosic materials undergo separation into a first portion and a second portion. In the illustrated embodiment, the lignocellulosic materials are separated into starch solution and fibers. It should be appreciated that fibers may include softwood fibers, hardwood fibers, groundwood fibers, energy crop fibers, or any other types of lignocellulosic fibers. It should further be appreciated that the lignocellulosic materials may undergo further separation into additional portions. For example, the fibers may be separated into different fiber types, as described in greater detail below.

As mentioned above, separation may include separation of starch from fibers (see, e.g., the illustrated embodiment of FIG. 1, as represented by block 130). Because starch is generally soluble in water, a starch solution is formed when liquid, such as water, is used to fiberize or wash the feedstock. Therefore, as seen in FIG. 1, a starch solution may be separated from the other materials, as represented by block 130, for example, by draining the waste water from the feedstock water. After separation, the starch may then be retained and re-combined with the fiber portion of the feedstock prior to subjecting the feedstock to enzymatic hydrolysis, as represented by block 170. In this manner, the starch can be converted to sugar along with the cellulose components of the feedstock. It should be appreciated, however, that the starch solution may be hydrolyzed separately from the other portions of the feedstock or diverted to a non-hydrolysis process or to a waste stream.

As represented by block 140 in FIG. 1, it should further be appreciated that the starch solution may optionally be concentrated prior to being re-combined with the fiber portion of the feedstock or prior to separate hydrolyzation. Suitable concentration methods may include but are not limited to evaporation methods and mesh or membrane technology, such as an ultrafiltration membrane, with a pore size suitable to retain the starch while allowing water to pass through.

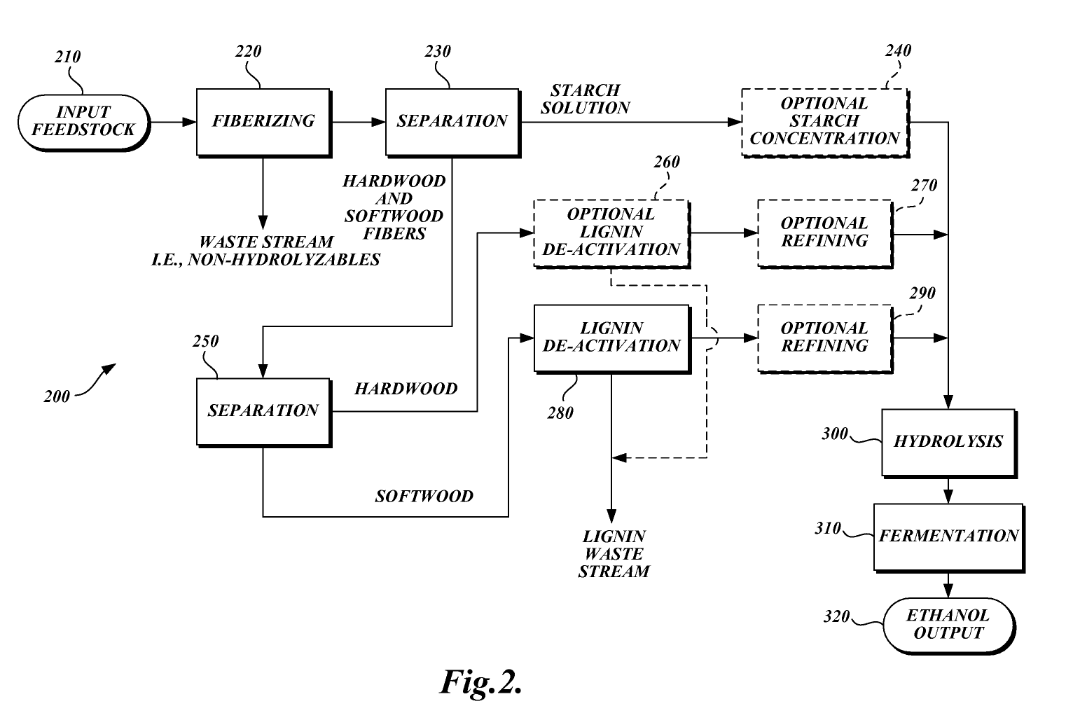

It should be appreciated that in addition to or in lieu of starch separation from fibers, different fiber types may also be separated from one another into first and second portions. Referring now to FIG. 2, a process diagram 100 for another method of converting lignocellulosic materials to ethanol is shown. It should be appreciated that the process shown in FIG. 2 is substantially similar to the process shown in FIG. 1, except for differences regarding fiber separation and processing. As a non-limiting example, hardwood and softwood fibers may be separated from one another, as represented by block 250 in FIG. 2, because such fibers may require different lignin deactivation processes for enhanced cost reduction and processing effectiveness. It should be appreciated that fiber separation may be performed by any suitable separation means including but not limited to fiber fractionation, for example, using pressurized rotating screens with screen baskets having round holes, as well as fiber density separation methods. As described in greater detail below, after being separated, the different fiber types may be subjected to different lignin deactivation processes depending on the fiber type.

In yet another embodiment of the present disclosure, separation may also include separation of groundwood fibers and/or fibers from one or more sources of energy crops, if present in a combined feedstock. These fibers also may require different lignin deactivation processes for enhanced cost reduction and processing effectiveness.

It should further be appreciated that fiber separation may include fractionating fibers into a long fiber fraction and a short fiber fraction and using the short fiber fraction for hydrolysis and ethanol production. The long fiber fraction from this process may be diverted to a non-hydrolyzing application, such as for use in papermaking or other suitable applications in which long fibers are preferred for their strength and bonding properties. The inventors have found that after such fractionation into long and short fiber fractions, higher levels of starch may be available for hydrolysis, because most of the original starch in the lignocellulosic materials can be retained and directed to the short fiber fraction.

As a non-limiting example of long/short fiber fractionation, the incoming fibers from an OCC feedstock collected may include from about 1.5 mm to 1.75 mm length weighted average fiber length (LWAFL), with a CSF of about 500-600. After fractionation, the short fiber fraction may include from about 1.2 mm to 1.5 mm LWAFL, with a freeness of about 350-500 CSF, and the long fiber fraction may include from about 1.6 mm to about 1.9 mm LWAFL, with a freeness of about 600-670 CSF.

After separation, at least one of the first and second portions of the lignocellulosic materials will be treated to remove and/or modify lignin present in the feedstock to make the fibers more responsive to enzymes during enzymatic hydrolysis. In the illustrated embodiment of FIG. 1, treatment or lignin deactivation is represented by block 150. In the illustrated embodiment of FIG. 2, treatment or lignin deactivation is represented by block 280 and optional block 260.

Suitable lignin deactivation processes include oxygen treatment in an alkaline medium, ozone treatment, other processes for modification and/or removal of lignin from the fibers, or any combination thereof. While not wishing to be bound by theory, it is believed that lignin deactivation processes allow for the use of reduced enzyme loading rates by opening up active sites for enzymatic hydrolysis and decreasing interactions of the enzymes with the lignin. In that regard, it is believed that lignin deactivation processes generally remove lignin present in the cellulose and/or modify the lignin to make it less likely to bond with the enzymes. Moreover, the alkaline medium is believed to alter the crystallinity of the cellulose structure to open up sites and make the cellulose generally more hydrolyzable.

In accordance with embodiments of the present disclosure, the fibers may be treated with oxygen in a suitable lignin deactivation process. For example, the fibers may be treated with oxygen in a pressurized vessel or standing pipe at a pressure from about 0 psi to about 120 psi, and more preferably from about 20 psi to about 120 psi, and at an elevated temperature from about 60 C to about 150 C, and more preferably 80 C to about 150 C.

In one suitable method, the oxygen treatment is carried out in an alkaline medium. As a non-limiting example, a suitable caustic agent is NaOH, used in the range of about 1% to about 20% by weight of the dried fiber, and more preferably in the range of about 1% to about 10% by weight of the dried fiber. The residence time for oxygen treatment may be in the range of about 5 to about 60 minutes.

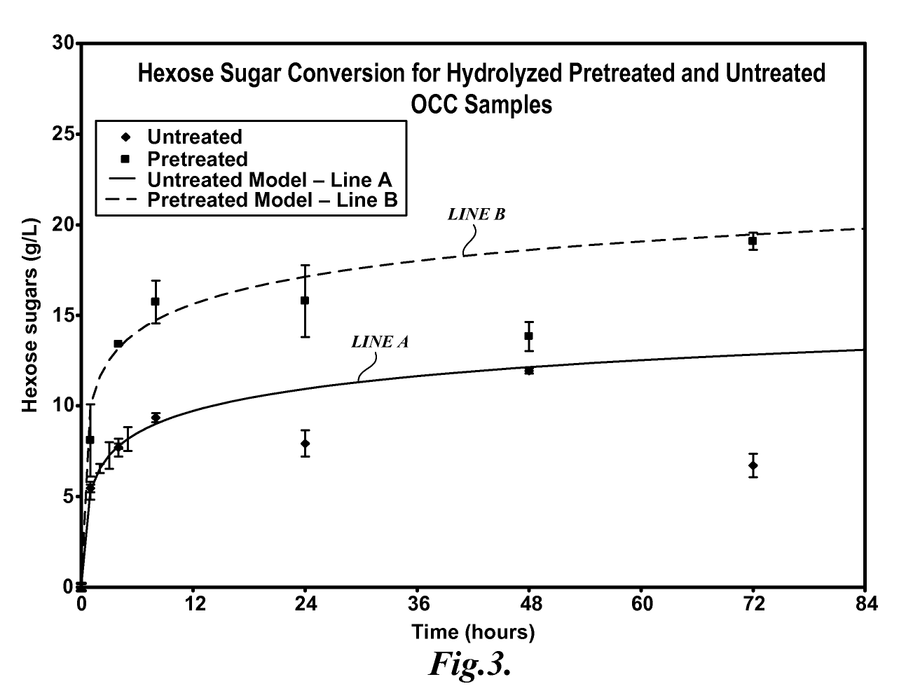

The inventors have found that multiple stages of oxygen treatment may be used to treat the fibers, for example, a process having two or more stages. As a non-limiting example, a suitable two-stage oxygen treatment process is as follows: stage 1 run with 6% caustic by weight of the dried fiber at 105 C for 1 hour, followed by stage 2 run with 2% caustic by weight of the dried fiber caustic at 105 C for 1 hour, resulting in a final pH of 11.7 and a final Kappa value of 40.9. The hexose sugar conversion results after this two-stage oxygen treatment process can be seen in FIG. 3 (see line B) comparatively with no treatment (see line A) over a 72-hour time period and are further described in EXAMPLE 4 below. The overall hexose sugar yield for the treated sample was nearly 100%. Due to the inclusion of lignin deactivation processing, the line B model for the conversion of starting material to sugar increases to about 20 g/L for the sample hydrolyzed with 20 FPU of enzymes per gram of OCC (about 97% conversion after 72 hours), a 51% increase over the results achieved with untreated OCC (line A). Because the near 100% conversion of sugar to ethanol via fermentation can be achieved, the yield of ethanol per ton of recovered paper is dependent upon conversion of cellulose, starch, and hemicellulose to sugar. Therefore, the results show that an oxygen/caustic lignin deactivation treatment for OCC increases the yield of ethanol produced (see line A) as compared to untreated OCC (see line B).

In accordance with other embodiments of the present disclosure, the fibers may be also treated with ozone for lignin deactivation, either alone or in addition to treatment with oxygen in an alkaline medium. Ozone was introduced as a bleaching chemical on an industrial scale in the beginning of the 1990s in order to achieve full pulp brightness without using chlorine-containing chemicals and is effective at both modifying and/or removing lignin from the fibers. As a non-limiting example of ozone use for fiber lignin deactivation, ozone can be added in a range of about 0.1% to about 2% ozone by weight of dried fiber. Ozone is typically produced on site through silent electrical discharge in a gas stream containing oxygen. Preferably, the feed gas is from water and organic compounds. Ozone may be applied to the fibers at a temperature from about 25° C. to about 60° C., at a pressure from about 10 psi to about 100 psi, and at a pH in the range of about 3 to about 5 by sulfuric acid addition. The ozone reaction is immediate, so retention time is not a factor.

In accordance with embodiments of the present disclosure, lignin deactivation of fibers with oxygen in an alkaline medium and/or ozone can lead to partially delignified fibers with a lignin reduction ranging from about 1% to about 50% reduction in Kappa level, and more preferably about 30% to about 50%, as compared to the Kappa level of the starting fibers (or lignocellulosic material). For example, a starting Kappa value range for OCC may be in the range of about 70 to about 120, or more preferably in the range of about 70 to about 90 according to a ten-minute Kappa test. A suitable reduction in Kappa value due to lignin deactivation is in the range of about 1 to about 60 points, and more preferably, about 20 to about 40 points.

A reduction in Kappa value can be indicative of lignin deactivation when the oxidized lignin dissolves into the caustic solution. However, it should be appreciated that a reduction in Kappa value is not required and is not always indicative of lignin deactivation. In that regard, lignins may be modified by the lignin deactivation process without dissolving into solution, which may improve the rate of enzymatic hydrolysis, but which may not be reflected in Kappa value reduction.

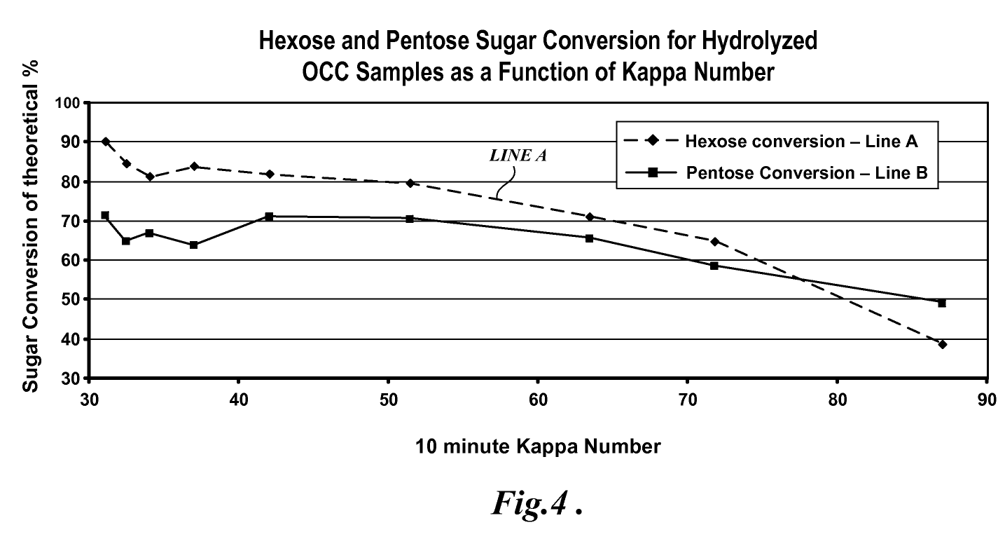

Although a reduction in Kappa value is not necessarily indicative of lignin deactivation, a reduction in Kappa value can be correlated to improved sugar conversion rates during hydrolysis. For example, FIG. 4 is a graph showing OCC conversion to hexose sugars (from cellulose) with data points connected as line A and pentose sugars (from hemicellulose), with datapoints connected as line B as a function of ten-minute Kappa number. This graph generally shows that a higher sugar conversion is achieved for both hexose and pentose sugars for lower Kappa numbers (or lignin content).

As mentioned above, different types of fibers require different lignin deactivation process steps. Therefore, the inventors have found that when fibers are separated into different types, the different fibers may be treated differently for lignin deactivation. For example, if softwood and hardwood portions are separated into first and second portions, as shown in FIG. 2, each portion may be treated differently for lignin deactivation. In a suitable oxygen treatment process, oxygen is generally run in excess, and the variables in the process may include caustic amount, residence time, temperature, and pressure. Caustic amount may varied for different types of fibers for suitable lignin deactivation. As non-limiting examples of suitable process conditions, an oxygen treatment process for lignin deactivation of softwood fibers may include about 2% to about 10% caustic per gram of dried fiber, and more preferably about 5% caustic per gram of dried fiber. On the other hand, an oxygen treatment process for lignin deactivation of hardwood fibers may include about 0% to about 10% caustic per gram of dried fiber, and more preferably about 0% to about 6% caustic per gram of dried fiber. An oxygen treatment process for lignin deactivation of energy crop fibers may include about 0% to about 10% caustic per gram of dried fiber, and more preferably about 0% to about 3% caustic per gram of dried fiber.

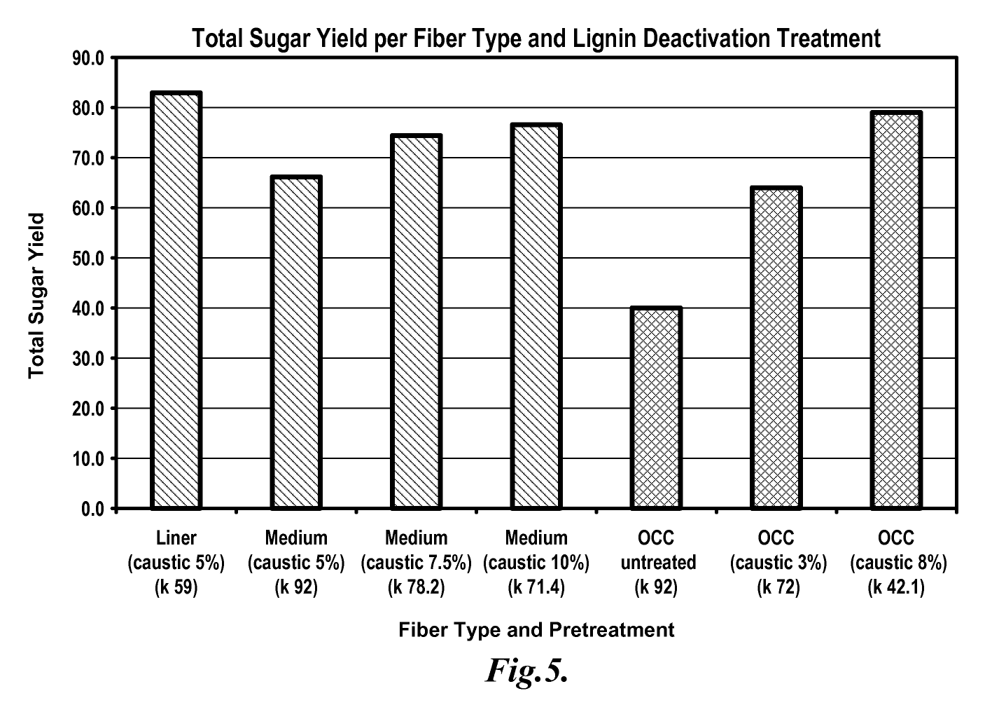

Referring to FIG. 5, the results in total sugar yield after enzymatic hydrolysis, as a function of fiber type and treatment variables, are shown. Three different lignocellulosic material types were used as samples: liner fibers (100% virgin softwood fibers), medium fibers (100% virgin hardwood fibers), and OCC (including non-virgin softwood fibers, hardwood fibers, and starch). In addition, different oxygen treatments were used on the various fiber samples, with end Kappa values and total sugar yields recorded for each fiber sample after hydrolyzing a 5% consistency sample of each with an enzyme loading of 20 FPU of enzymes per gram of fiber. The experimental conditions for these results are further described in EXAMPLE 5 below.

The results show that different treatments may provide optimal results for lignin deactivation in softwood, hardwood, and mixed OCC samples. In that regard, softwood samples can be processed with about 5% caustic, and hardwood samples (which have much higher starting Kappa levels) hardwood pulp samples are optimally processed with an amount of caustic that will yield high amounts of sugar in hydrolysis balanced against chemical costs and carbohydrate losses occurring in the pretreatment. In one embodiment, the amount of caustic for hardwood pulp samples may be about 5-10%. However, it is possible that lower levels of caustic may be effective for hardwood treatment because hardwood is generally less recalcitrant overall than softwood. As seen in FIG. 5, for instance, at a Kappa level of 92, the hardwood fibers have a much higher sugar conversion than OCC, also at a Kappa level of 92. Therefore, by using different amounts of caustic in each portion, less total caustic to process a combined softwood and hardwood feedstock can be reduced by separating the softwood from the hardwood. This translates into lower chemical usage and a higher overall yield because higher caustic results in more carbohydrate degradation in addition to lignin removal and deactivation.

Moreover, the results show that high sugar yields can be achieved for 100% hardwood samples, even with a Kappa value that is relatively higher than the Kappa value achieved for 100% softwood samples. This means that ultimate treatment of the hardwood fiber from recovered paper, like OCC, depends on the as-received Kappa level of the hardwood fibers, which is generally significantly higher than the as-received Kappa level of the softwood fibers because hardwood is generally processed less than softwood in the papermaking process.

Oxidized lignins generally dissolve in a caustic solution and can therefore be washed out of lignocellulosic material in a waste stream. Therefore, it should be appreciated that embodiments of the present disclosure may include wash stages to wash out and recover caustic solution and lignins. It should further be appreciated that suitable neutralization stages may be incorporated into the processes as needed.

In addition to lignin deactivation, the feedstock may further be subjected to a comminution process, for example, by refining or mechanical agitation, either prior to or simultaneously with enzymatic hydrolysis. When comminuted prior to enzymatic hydrolysis, it should be appreciated that such comminution may be either together with or separate from the lignin deactivation process. It should further be appreciated that suitable comminution generally results in more comminuted fibers and may be accomplished by refining, static mixing, propeller mixing, or any other suitable mechanical agitation of the lignocellulosic stock. Such comminution processes are generally designed to increase the surface area on the fibers to allow for increased mass transfer of enzymes to the fibers during hydrolysis.

In the illustrated embodiments of FIGS. 1 and 2, the methods include optional refining comminution processes, as represented by block 160 in FIG. 1 and blocks 270 and 290 in FIG. 2. Generally described, a fiber refiner generates an intense mechanical action which collapses the fibers and shreds up (fibrillates) the outside of the fibers, exposing more fiber surface area. Increasing the surface area of the lignocellulosic fibers effectively opens up active sites on the fibers to improve the mass transfer of the enzymes to the lignocellulosic fiber surfaces. In the paper industry, refining is traditionally performed by passing the pulp slurry between rotating plates covered with bars. The shearing action between the plates causes the fibers to flex and release. After many cycles of this action, the fibers collapse and become fibrillated.

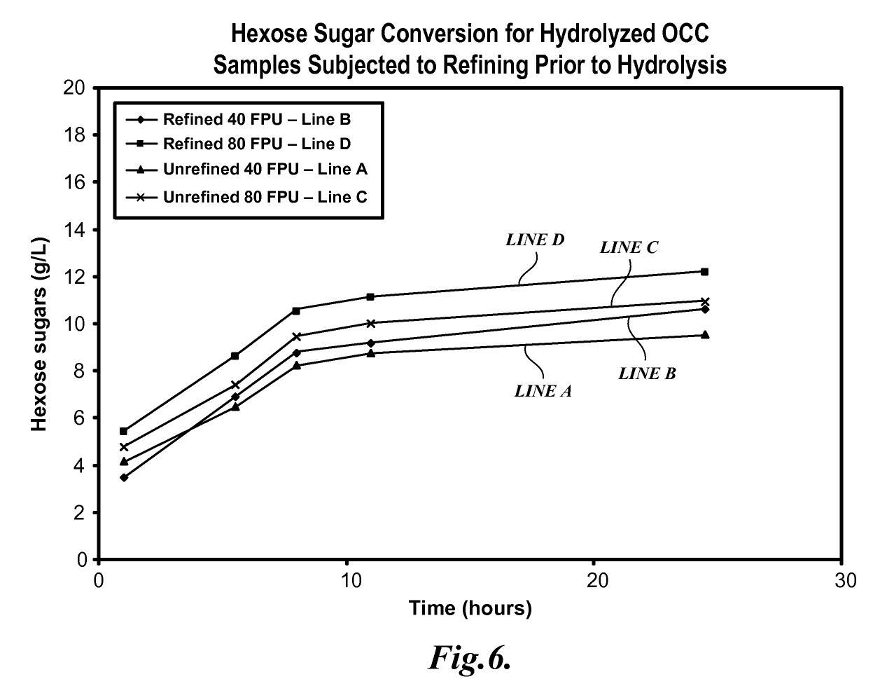

As a non-limiting example, FIG. 6 shows a graph of hexose sugars in g/L versus time in hours of an OCC samples hydrolyzed with, respectively, 40 and 80 FPU of enzymes per gram OCC and with no lignin deactivation processing, but with refining at 100 psi and 163 C prior to hydrolysis. Due to the pre-hydrolysis refining process, the conversion of starting material to sugar increases about 11% for the sample hydrolyzed with 40 FPU of enzymes per gram OCC and about 12% for the sample hydrolyzed with 80 FPU of enzymes per gram OCC. The pre-hydrolysis refining was fun at 950 kw-hr/OD ton of OCC.

In accordance with one embodiment of the present disclosure, as a result of the comminution process, the fibers are modified to have a reduced level of freeness as compared to fibers prior to the lignin deactivation process. As a non-limiting example, fibers from OCC prior to the lignin deactivation process may have a Canadian Standard Freeness (CSF) value of from about 500 to 600 CSF, and after the refining process, the fibers have from 50 to 400 CSF. In addition, the water retention value (WRV) of the modified fibers may increase to less than about 3 g water/g fiber. The refining process may be carried out under a pressure in the range of about 0 psi to about 150 psi, and more preferably in the range of about 50 psi to about 120 psi, and at a consistency in the range of about 2% to about 30%, preferably in the range of about 8% to about 25%.

A lignin deactivation process may be combined with a suitable comminution process, either together in one process stage or in subsequent process stages prior to enzymatic hydrolysis. While not wishing to be bound by theory, it is believed that the combination of a lignin deactivation process with a comminution process allows for the use of reduced enzyme loading rates by opening up active sites for enzymatic hydrolysis and decreasing interactions of the enzyme with lignin. In addition, as described in greater detail below, a comminution process may also be combined with enzymatic hydrolysis for improved mass transfer of enzymes during the hydrolysis process.

After suitable lignin deactivation, the other streams of feedstock can be recombined with the activated streams for hydrolysis, which is represented by block 170 in FIG. 1 and block 300 in FIG. 2.

Enzymatic hydrolysis will now be described in greater detail. The treated fibers and the starch solution are hydrolyzed with an enzyme mixture to produce a hydrolysate, as represented by block 170 in the exemplary process diagram of FIG. 1 and by block 300 in the exemplary process diagram of FIG. 2. The hydrolysis mixture may include at least one of cellulose, hemicellulose, and starch. The enzymatic hydrolysis can be carried out for a sufficient time to catalyze the hydrolysis of cellulose, hemicellulose, and starch to form a combination of smaller oligosaccharides, disaccharides (cellobiose), glucose, amylose, dextrose, xylose, arabinose, mannose, and galactose in the hydrolysate.

The hydrolysis mixture is placed in a suitable medium and contacted with an enzyme mixture. The enzymatic hydrolysis of lignocellulosic stock can be carried out at suitable conditions, such as a suitable pH and temperature, depending on the specific enzymes being used in the process. For example, the fibers may be neutralized and washed prior to enzymatic hydrolysis. Further, the fibers may be pH adjusted to an acid pH, for example, in the range from about 4.5 to about 5.5, and preferably 4.8 to 5.0. Moreover, the fibers may be temperature adjusted to a temperature range from about 40° C. to about 60° C. In one suitable embodiment, as described in greater detail below, the enzyme mixture may be blended directly into a fiber refiner before entry into the hydrolysis reactor vessel.

The enzyme mixture used in enzymatic hydrolysis may include at least one cellulose hydrolyzing enzyme, such as cellulase (or a mixture of cellulases). The enzyme mixture may further include at least one starch hydrolyzing enzyme, such as amylase (or a mixture of amylases). In some embodiments, the enzyme mixture further includes at least one xylan hydrolyzing enzyme, such as xylanase (or a mixture of xylanases). The hydrolyzing enzymes (e.g., cellulase(s), xylanase(s), and amylase(s)) may be provided in the form of purified or partially purified enzymes or as a mixture.

In accordance with embodiments of the present disclosure, the enzyme mixture comprises at least one amylase at a total enzyme load of from 0.01 to 100 starch solid units (SSU)/g fiber. Further in accordance with embodiments of the present disclosure, the enzymes are at a total enzyme load of less than about 100 FPU of enzymes per gram lignocellulosic material is used to produce the hydrolysate. In another embodiment the enzymes are at a total enzyme load of less than about 50 FPU of enzymes per gram lignocellulosic material is used to produce the hydrolysate. In one embodiment, the enzymes are at a total enzyme load from about 2 to about 30 FPU enzymes per gram lignocellulosic material is used to produce the hydrolysate. An additional enzyme load of Beta-glucosidase can be used at a cellulase to Beta-glucosidase ratio of about 1 to 5 to prevent any feedback inhibition caused by cellobiose accumulation.

The hydrolysate, comprising glucose, other 6-carbon sugars, xylose, and other 5-carbon sugars, is converted to ethanol by fermentation material, as represented by block 180 in the exemplary process diagram of FIG. 1 and by block 310 in the exemplary process diagram of FIG. 2. As mentioned above, it should be appreciated that residual products may be produced from the fermentation process, such as yeast, lignin, and other non-fermented products. In one embodiment, the fermentation material comprises a microorganism capable of fermenting glucose and xylose to ethanol.

As used herein, the term “fermentation materials” may include any material or organism capable of producing ethanol. While not to be construed as limiting, the term includes bacteria, such as Zymomonas mobilis and Escherichia coli; yeasts such as Saccharomyces cerevisiae or Pichia stipitis; and fungi that are natural ethanol-producers. Fermentation materials also includes engineered organisms that are induced to produce ethanol through the introduction of foreign genetic material (such as pyruvate decarboxylase and/or alcohol dehydrogenase genes from a natural ethanol producer). The term further includes mutants and derivatives, such as those produced by known genetic and/or recombinant techniques, of ethanol-producing organisms, which mutants and derivatives have been produced and/or selected on the basis of enhanced and/or altered ethanol production.

In accordance with embodiments of the present disclosure, the enzymatic hydrolysis and fermentation processes may be carried out simultaneously, i.e., simultaneous enzymatic hydrolysis (or saccharification) and fermentation, such that the lignocellulosic stock is treated with at least a hydrolyzing enzyme and a microorganism capable of converting the hydrolysate to ethanol, in the same medium and under the same conditions.

Saccharomyces cerevisiae may be used to ferment glucose to produce ethanol in a simultaneous saccharification and fermentation reaction. However, any suitable organism capable of converting glucose to ethanol may be used in accordance with embodiments of the present disclosure, such as Kluveromyces species and/or any yeast that has been genetically engineered or selected on the basis of its ability to grow and ferment glucose to produce ethanol.

Ethanol is recovered from the fermentation using known methods. As used herein, the term “ethanol” includes ethyl alcohol or mixtures of ethyl alcohol and water. In accordance with embodiments of the present disclosure, ethanol recovery may further include concentrating the ethanol to provide fuel grade ethanol. In one embodiment, the ethanol can be concentrated via distillation to a water and ethanol azeotrope. In another embodiment, the ethanol azeotrope can be further distilled to produce a fuel grade ethanol. In yet another embodiment, molecular sieves can be used to remove water molecules from the water and ethanol azeotrope and produce a fuel grade ethanol. In yet another embodiment, the water and ethanol azeotrope can be further concentrated to a fuel grade ethanol by filtration with suitable membrane.

Various embodiments of the present disclosure will produce a yield of ethanol at least 80 gal/ton of recovered paper on a bone dry ton basis. In another embodiment, the method produces a yield of ethanol at least 100 gal/ton of recovered paper on a bone dry ton basis. In yet another embodiment, the method produces a yield of ethanol at least 120 gal/ton of recovered paper on a bone dry ton basis. In yet another embodiment, the method produces a yield of ethanol at least 140 gal/ton of recovered paper on a bone dry ton basis. Conversion of starch to ethanol may contribute from about 5 to about 10 gal/ton to the yield.

In accordance with other embodiments of the present disclosure, a composition for enzymatic hydrolysis is provided. The composition generally includes fibers derived from lingnocellulosic materials, starch, and hydrolyzing enzymes, including but not limited to cellulase, xylanase, and amylase. In one suitable embodiment, the composition includes from about 0.5% to about 15% of starch in the solid mixture. In one embodiment, the composition comprises greater than about 60% or more preferably in the range of about 70% to about 99% of fibers derived from lignocellulosic materials in the solid mixture. The amount of enzymes in the composition is less than about 100 FPU of enzymes per gram lignocellulosic material, less than about 50 FPU of enzymes per gram lignocellulosic material, or in the range of about 2 to about 30 FPU of enzymes per gram lignocellulosic material. The composition may be used in the methods of the present disclosure described herein.

As mentioned above, in addition to comminution of the feedstock prior to hydrolysis, the inventors have further found that in order to overcome enzyme mass transfer limitations the lignocellulosic stock can be comminuted, such as mechanically agitated, mixed, and/or refined, during the enzymatic hydrolysis process and/or the turbulence of the stock undergoing enzymatic hydrolysis can be increased to increase the rate of transport of enzymes to and from the lignocellulosic fibers. Therefore, embodiments of the present disclosure are generally directed to systems and methods for enzymatic hydrolysis of lignocellulosic materials subjected to simultaneous comminution. Suitable comminution generally results in more comminuted fibers having increases surface area.

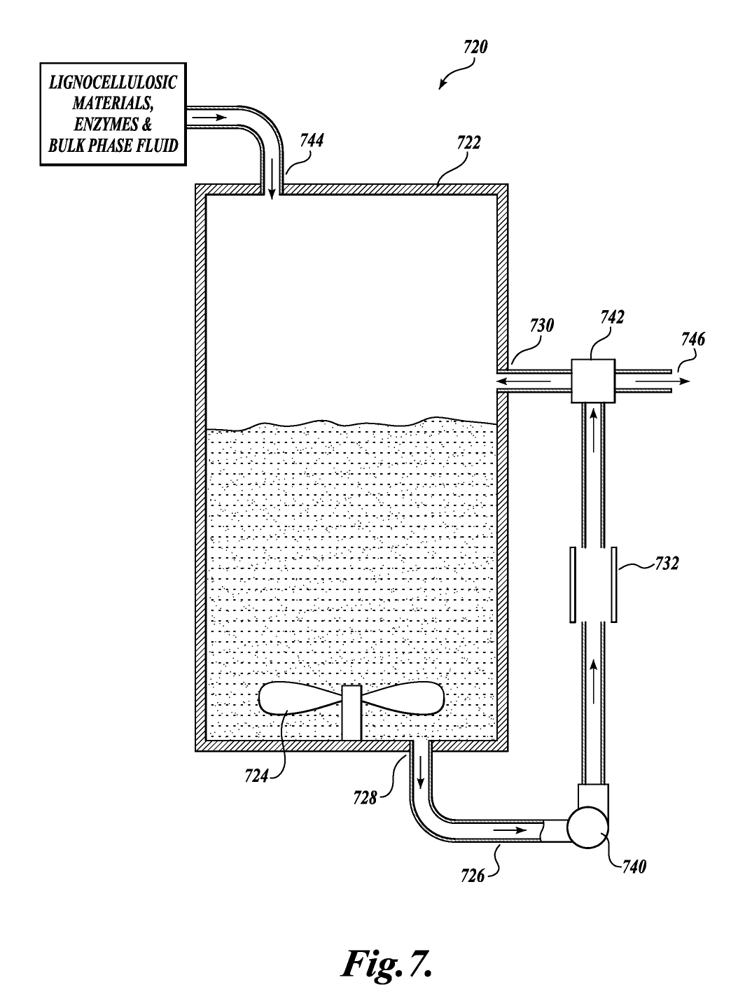

Referring to FIG. 7, there is shown a process diagram for an enzymatic hydrolysis system designed in accordance with one embodiment of the present disclosure. The system 720 generally includes a reactor vessel, such as a tank 722, configured to receive a mixture of lignocellulosic stock, enzymes, and bulk phase fluid (typically water). The tank 722 includes a first agitator 724 for mixing within the tank 722. The system further includes a recycle loop 726 extending from and returning to the tank 722, and a second agitator or comminutor 732 to promote additional mixing in the recycle loop 726. As described in greater detail below, enzymatic hydrolysis rates can be improved by mixing in the recycle loop 726 to generate a higher turbulence intensity than is generated in the reactor vessel, for example, to achieve micro-turbulence in the recycle loop 726, without having the deleterious effect of deactivating the enzymes or the enzyme mixture.

As mentioned above, enzymatic hydrolysis is limited by the mass transfer limitations of the enzymes. To overcome enzyme mass transfer limitations, the lignocellulosic stock can be comminuted, agitated, mixed, and/or refined during the enzymatic hydrolysis process and/or the turbulence of the stock undergoing enzymatic hydrolysis can be increased to increase the rate of transport of enzymes to and from the lignocellulosic fibers. As described in greater detail below, the embodiments of the present disclosure accomplish one or both of these objectives by incorporating a second agitator 732 (such as a high shear mixing and agitation device or a refiner) that comminutes the lignocellulosic stock and/or creates a turbulent environment in the recycle loop 726 of the system 720. It should be appreciated that the second agitator 732 is a device capable of comminuting the lignocellulosic stock, including but not limited to a refiner, static mixer, propeller mixer, or any other suitable mechanical agitation device.

In an enzymatic hydrolysis system, more than one reactor vessel or tank may be used to process the lignocellulosic stock. Further in that regard, the system may be run in a batch or continuous process, with the plurality of vessels connected in either parallel or series. As non-limiting examples, a batch system may be run with reactor vessels in parallel with one another, and a continuous system may be run with reactor vessels in series with one another. Each reactor vessel or tank 722 is generally comminuted by an agitator 724, such as a propeller mixer as seen in the illustrated embodiment of FIG. 7, to maintain the lignocellulosic stock in the tank suspended in a mixture and to mix the entire tank volume with minimum processing costs.

It should be appreciated that one or more vessels suitable for the processes described herein may be of varying dimensions, designed to hold varying capacities, depending on the economics of a particular system. It should further be appreciated, however, that the economic analysis for system design may include, but is not limited to, the following factors: the number and size of the tanks, the power requirements for agitation of the tanks, and the consistency of the stock in the tanks. For example, the required tank volume for a process may decrease if stock consistency is high, while at the same time, the power requirement may increase to maintain total tank agitation for stock at a higher consistency. Typical enzymatic hydrolysis processes run in the range of about 2% to about 30% stock consistency. In one embodiment the stock consistency is about 8% to about 25%. In another embodiment, the consistency is above 12%.

The inventors have found, however, that optimal consistency may vary depending on the type of feedstock. A higher cellulose concentration in the feedstock can be hydrolyzed to a higher concentration of sugar and ultimately a higher concentration of ethanol. The greater the cellulose concentration of the feedstock, the lower the stock consistency can be and still result in an ethanol concentration that can be economically distilled. Optimal enzymatic hydrolysis tank size will vary depending on plant size, but may vary from about 40,000 gallons to about 200,000 gallons. In some cases, however, the tank size may be up to about 2.5 million gallons.

Due at least in part to power requirements, it is difficult to achieve adequate turbulence in a large tank for full mixing and effective mass transfer of enzymes to the surfaces of the lignocellulosic fibers. Further adding to this problem is the nature of the stock itself. In that regard, aqueous slurries of lignocellulosic stock in a tank tend to dampen tank turbulence as a result of the tendency of the lignocellulosic stock to flocculate, especially when the stock has a high consistency. (As hydrolysis progresses and the apparent viscosity of the stock decreases, the turbulence in the tank tends to increase.) Therefore, the turbulence in large agitated tanks is typically inadequate to substantially increase the rate of enzymatic hydrolysis. Even in a large agitated tank with adequate macro-turbulence to achieve full mixing in the tank, the micro-turbulence required for effective mass transfer of enzymes to the lignocellulosic stock may be absent in most or all of the tank.

In addition, due to the inefficiencies of mixing in large agitated tanks, as described above, the shear stress created by violent mixing in a large tank tends to break down and deactivate the enzymes by irreversibly altering the tertiary structures of the enzymes. The level of mixing required on a smaller scale (for example, turbulence in a recycle loop having enough flow to allow a 1 to 4 hour turnover time in a single tank), however, results in lower shear stress on the lignocellulosic stock than the mixing required in the large tank. Therefore, more vigorous mixing of a smaller portion of the lignocellulosic stock, for example, in a recycle line, to achieve suitable micro-turbulence improves enzyme mass transfer without having the effect of deactivating the enzymes.

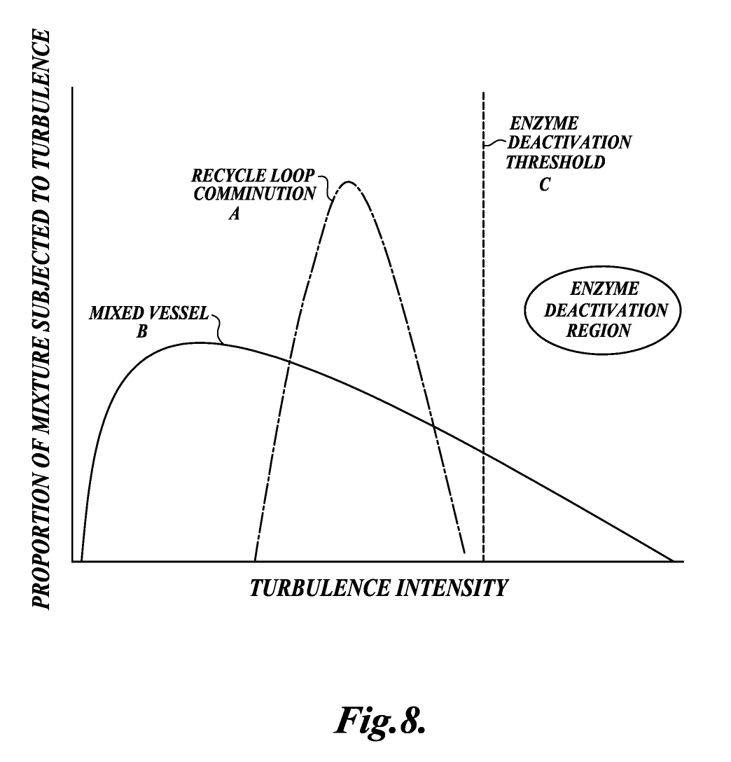

Referring to FIG. 8, a graph of turbulence intensity versus the proportion of lignocellulosic stock subjected to turbulence is shown. This graph indicates that the agitation for comminution in the recycle loop (line A) can be run at a higher intensity than the agitation for comminution in a mixed vessel (line B) without reaching a level of turbulence intensity that deactivates the enzymes. So long as the turbulence intensity imposed by the agitator in the recycle loop is below the deactivation threshold (line C) for a specific enzyme, then agitation in the recycle loop promotes increased enzyme transfer to and from the lignocellulosic fibers for an improved rate of overall enzymatic hydrolysis of the stock. With reference to line B, a much smaller portion of lignocellulosic stock is activated at the same intensity in a system employing mixing in the large vessel to achieve improved enzyme mass transfer.

While it should be appreciated that high turbulence intensity may deactivate enzymes resulting in low lignocellulosic stock to sugar conversion rates, it is further believed that excessive comminuting of the lignocellulosic stock also may lead to low sugar conversion rates. In that regard, it should be appreciated that feedstock consistency can be controlled to prevent over-comminuting. Moreover, agitation can be intermittent or pulsed, for example, 15 minutes of agitation on and 15 minutes of agitation off, to further prevent over-comminuting.

Energy crops, including but not limited to perennial plant species such as switch grass, are believed to be easily subjected to over-comminuting due to their more fragile nature. Therefore, a feedstock that is easily comminutable, such as an energy crop feedstock, can also be subjected to intermittent or pulsed agitation, for example, 15 minutes of agitation on and 15 minutes of agitation off, to further prevent over-comminuting. In addition, feedstock consistency can be controlled to prevent over-comminuting.

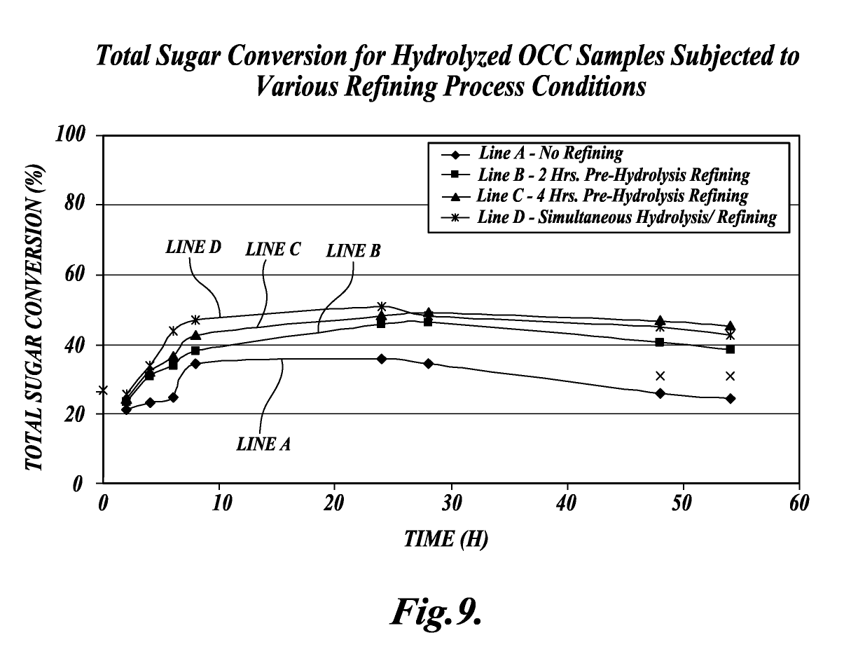

While not wishing to be bound by theory, it is believed that a simultaneous hydrolysis and comminution process continuously opens up active sites for enzyme hydrolysis, while a pre-hydrolysis comminution process may only have limited success because open sites are filled by enzymes after the agitation has stopped. Referring to FIG. 9, experimental results of total sugar conversion versus time for Samples A-D under various refining conditions are shown. Using a Valley Beater lab refiner having a recycle loop, 2% consistency OCC feedstock was run at four sample conditions, Samples A (no refining), Samples B and C (refining prior to enzymatic hydrolysis, 2 hours and 4 hours, respectively), and Sample D (simultaneous refining and enzymatic hydrolysis). The experimental conditions for these results are further described in EXAMPLE 8 below.

As seen in FIG. 9, Sample D (simultaneous refining and enzymatic hydrolysis) showed a greater increase in sugar conversion rate than the other samples, Sample A (no refining) and Samples B and C (refining prior to enzymatic hydrolysis, 2 hours and 4 hours, respectively) over time and a higher sugar conversion rate than the other samples at 24 hours. Samples B and C (refining prior to enzymatic hydrolysis) showed a greater increase in sugar conversion rate than Sample A (no refining) over time, with Sample C (4 hours refining prior to enzymatic hydrolysis) showing a greater increase over time than Sample B (2 hours refining prior to enzymatic hydrolysis).

The operation of the system 720 will now be described in greater detail. Returning to FIG. 7, feedstock (for example, lignocellulosic stock, enzymes, and bulk phase fluid) is fed to the tank at system inlet 744 and removed at system outlet 746. The feedstock is mixed in the tank by propeller mixer 724 and recycled by recycle loop 726. In that regard, the recycle loop 726 includes a pump 740 to pump stock from a recycle loop inlet 728 at a bottom portion of the tank 722 to a recycle loop outlet 730 at or near a top portion of the tank 722. In this configuration, the recycle loop 726 promotes additional tank mixing from the bottom portion of the tank 722 to the top portion of the tank 722. However, it should be appreciated that the loop may extend from and return to ports located in any position on the tank 722. It should further be appreciated that multiple ports, whether inlets or outlets, for the same recycle loop or multiple recycle loops are within the scope of the present disclosure.

The second agitator 732 is disposed within the recycle loop 726 to promote mixing in the recycle loop 726. Therefore, the pump 740 in the recycle loop 726 pumps a side stream of stock from the agitated tank 722 to the recycle loop 726, where the stock is subjected to intensified comminution or agitation in the recycle loop 726 and then recirculates the side stream back to the tank 722. In a continuous system, splitter 742 sends a portion of the side stream to the system outlet 746 and a portion of the side stream back to the tank 722. It should be appreciated that in a batch system, no stock is sent to the system outlet 746 until the enzymatic hydrolysis step has been completed.

In the illustrated embodiment of FIG. 7, the second agitator 732 is a fiber refiner. Generally described, a fiber refiner generates an intense mechanical action which collapses the fibers and shreds up (fibrillates) the outside of the fibers, exposing more fiber surface area. Increasing the surface area of the lignocellulosic fibers effectively opens up active sites on the fibers to improve the mass transfer of the enzymes to the lignocellulosic fiber surfaces. In the paper industry, refining is traditionally performed by passing the pulp slurry between rotating plates covered with bars. The shearing action between the plates causes the fibers to flex and release. After many cycles of this action, the fibers collapse and become fibrillated.

Refiners are advantageous agitators or comminutors suitable for the systems described herein because refiners have several control variables, by which mixing intensity and turbulence can be adjusted to prevent enzyme deactivation. In that regard, the refiner control variables include the following: refiner plate design, flow rate through refiner plates, speed of the plates, and the gap between the plates. Adjustments to these variables help control the turbulent environment such that the activity of the enzymes is optimized. Other variables affecting refining include stock consistency and the pressure at which the refiner is maintained. As a non-limiting example, the refining process can be carried out under a pressure in the range of about 0 psi to about 150 psi for a feedstock consistency of about 1 to about 30 percent.

In the illustrated embodiment, the refining process takes place in a recycle loop within the enzymatic hydrolysis system. However, it should be appreciated that in other embodiments, a refining process may be a fiber preparation step that takes place prior to enzyme addition or prior to the entry of the lignocellulosic feedstock into the enzymatic hydrolysis system, either in lieu of or in addition to a refiner loop in the enzymatic hydrolysis system. It should further be appreciated that the feedstock may be subjected to other lignin deactivation processes, such as oxygen/alkaline and/or ozone treatment, pre-hydrolysis comminution, or other processing prior to being fed into the reactor, as described in detail above.

As a result of a refining process, lignocellulosic fibers are modified to have a reduced level of freeness as compared to lignocellulosic fibers prior to refining. As a non-limiting example, fibers from OCC prior to refining may have a Canadian standard freeness (CSF) value of from about 500 to 600 CSF, and after the refining process, the fibers have from 50 to 400 CSF. In addition, the water retention value (WRV) of the modified fibers may increase from 1.2 g water/g fiber to about 2.2 g water/g fiber.

In the illustrated embodiment of FIG. 7, feedstock components (lignocellulosic materials, enzymes, and bulk phase fluid) are added together at the system inlet 744 at the top of the tank 722. It should be appreciated, however, that the feedstock components (lignocellulosic materials, enzymes, and bulk phase fluid) may be added individually or together at any location in the system 720, for example, immediately preceding or immediately following the second agitator 732 in the recycle loop 726. As a non-limiting example, some or all of the feedstock components may be added immediately preceding the second agitator 732 in the recycle loop 726. As another non-limiting example, lignocellulosic stock and bulk phase fluid may be added at the inlet at the top of tank, while enzymes are added immediately preceding the second agitator 732. As another non-limiting example, the enzymes may be added at the inlet at the top of tank, while lignocellulosic stock and bulk phase fluid are added immediately preceding the second agitator 732.

As yet another non-limiting example, in a continuous system, a tank farm is operated as a train, with each stage discharging into the next in sequence and having subsequent secondary agitators in-line between the stages. It should be appreciated that in such configurations the recycle loops are feed loops into the following stage. It should further be appreciated that in such continuous systems, secondary agitation in-line between the stages may not be required in the later stages of the system as a result of decreasing apparent viscosity as the stock travels from stage to stage. As stock apparent viscosity decreases, tank agitation alone may be adequate to activate the stock and enhance enzyme mass transfer in the later stages.

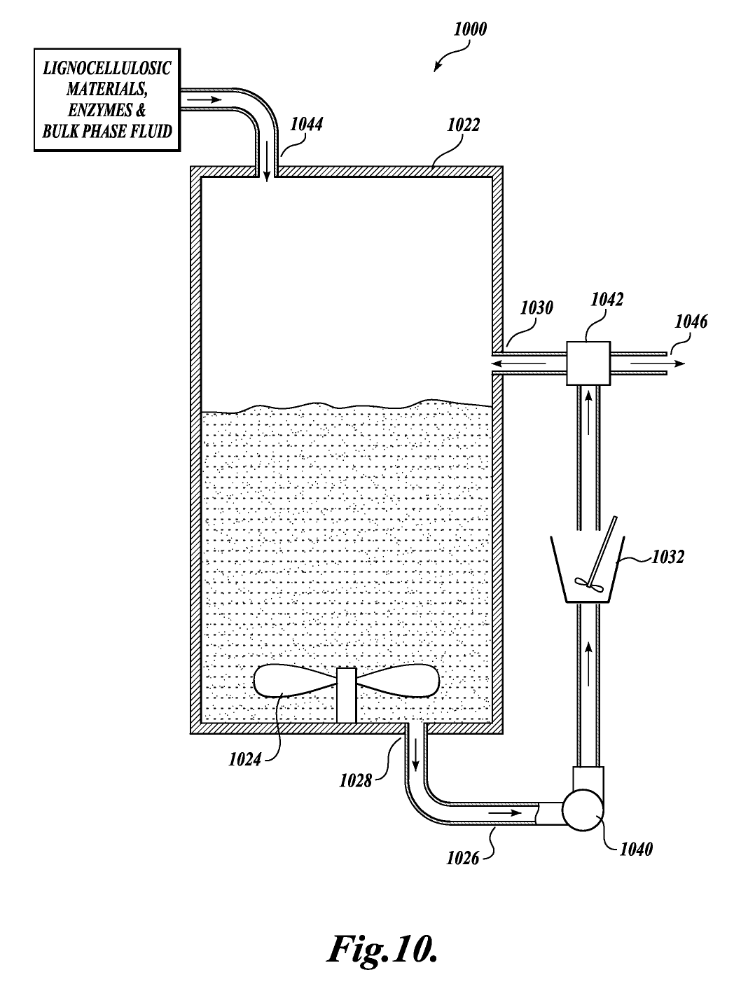

Now referring to FIG. 10, another embodiment of the present disclosure will be described in greater detail. It should be appreciated that the following system is substantially identical in materials and operation as the previously described embodiment, except for a difference regarding the second agitator. For clarity in the ensuing descriptions, numeral references of like elements of the system are similar, but are in the 1000 series for the illustrated embodiment of FIG. 10.

As seen in FIG. 10, the second comminutor or agitator 1032 is a smaller mixing tank run in the recycle loop 1026 from and returning to the tank 1022. Like the refiner 732 described above, the mixing tank 1032 is also designed to promote suitable mixing in the recycle loop 1026 for increased enzyme mass transfer to and from the surfaces of the lignocellulosic fibers and improved rates of enzymatic hydrolysis of the lignocellulosic stock. It should be appreciated that in other embodiments of the present disclosure, the second agitator may be a static mixer, a nozzle, or any other suitable mechanical agitation or mixing device.

It should be appreciated that advantages of the systems and methods described herein include decreased vessel residence time for the enzymatic hydrolysis reaction, resulting in higher volumes and/or reduced capital required for plant design and increased enzyme efficiency, resulting in savings relating to decreased enzyme use. The following examples provide process conditions for batch and continuous enzymatic hydrolysis systems and describe results achieved by the respective systems.

Non-limiting examples of the methods described herein are provided below. Examples 1-8 demonstrate the feasibility of the systems and methods systems and methods for producing a hydrolysate from lignocellulosic materials. It should be appreciated that, while the following examples illustrate methods for practicing embodiments of the invention, they should not be construed to limit the scope of the present disclosure.

Examples 1-3 demonstrate representative methods for enzymatic hydrolysis of pulp derived from recovered paper, fermentation of a hydrolysate from pulp samples derived from recovered paper, and analyzing sugar and ethanol produced from recovered paper, respectively. Example 4 provides a comparative analysis of sugar yield from treated and untreated OCC samples, wherein the treated sample is treated with an oxygen/NaOH lignin deactivation treatment. Example 5 provides a comparative analysis of sugar yield from various fiber types subjected to various lignin deactivation treatments. Examples 6 and 7 demonstrate exemplary lignocellulosic ethanol plants and describes their capacities. Example 8 provides a comparative analysis of sugar yield from various OCC samples subjected to various refining conditions.

### EXAMPLE 1

### Representative Method for Enzymatic Hydrolysis

This Example shows a representative method for enzymatic hydrolysis of pulp derived from recovered paper.

Methods:

Enzymatic hydrolyses were conducted in a 50 mL volume in 100 mL sealed, screw-capped Erlenmeyer flasks. The pulp concentration was 2% weight of fiber per weight of water, and the hydrolysis medium was 0.05 M acetate buffer, pH 4.8. The flasks were incubated at 50° C. in a New Brunswick Scientific Environmental Shaker at 150 rpm for 1 hour prior to addition of the enzymes to allow the temperature to stabilize at the reaction temperature. Beta-glucosidase and cellulase were then added using a micropipette to yield the desired enzyme loading. Ten to twenty FPU of enzymes per gram of fiber were used in the experiments. Sufficient β-glucosidase activity was added in all cases to boost the β-glucosidase:FPU activity ratio to 5:1.

Enzymes. Iogen Inc. (Ottawa, Ontario, Canada) supplied DP-140, a commercial cellulase mixture with an activity of 60 filter paper units (FPU) per mL and 112 CBU per mL or a commercial cellulase system (Celluclast) from Novozymes (NC. USA) with an activity of 80 FPU/ml and 50 CBU/ml β-glucosidase activity was used. To increase β-glucosidase activity and to prevent feedback inhibition, Novozyme 188 (Novozyme, N.C., USA), a β-glucosidase solution with an activity of 490 CBU per mL, was used.

### EXAMPLE 2

### Representative Method for Fermentation of Hydrolysate

This Example shows a representative method for fermentation of hydrolysate from pulp samples derived from recovered paper.

Methods:

Yeast. The yeast used was a commercially available strain of Saccharomyces cerevisiae (strain K), originally obtained from Lallemand (Montreal, Quebec, Canada).

Preparation of Inoculum. Batch Experiments were Initiated with a Starter Culture to insure uniform yeast concentration and characteristics in all of the shake flasks. Starter cultures were prepared by transferring a loop of yeast from an agar plate to 50 mL yeast extract-peptone-glucose broth (1% w/v yeast extract, 2% w/v peptone, and 2% w/v glucose) in a foam plugged 125 mL Erlenmeyer flask, and incubating overnight at 30° C., 150 rpm to late exponential phase. This starter culture was then used to inoculate 6 new flasks of the same medium, and these flasks were again grown up to exponential phase overnight. The yeast was then harvested from all flasks by centrifugation and re-suspended to a 50 mL volume in distilled water. Two mL of this concentrated inoculum was used to inoculate each experimental culture.

Fermentation. Fermentations were conducted in sealed 50 mL serum vials (40 mL working volume) incubated at 30° C. and 150 rpm. Fermentation conditions were adjusted to minimize yeast growth and thereby produce a value for the maximum alcohol yield. Growth was minimized by: (1) conducting the fermentations under microaerophilic reaction conditions, and (2) using a very large yeast inoculum. No nutrients were added to the fermentation medium.

Samples (1.5 mL) were withdrawn at the indicated times, and centrifuged (13,000 g, 3 minutes) to remove yeast. The samples were frozen until analysis.

### EXAMPLE 3

### Methods for Analyzing Sugar and Ethanol Produced

This Example shows a representative method for analyzing the sugar and ethanol produced from recovered paper in accordance with various embodiments of the methods of the invention.

Methods:

Ethanol and sugar analysis. Ethanol was analyzed by direct injection into a Varian 3800 GC (Varian Inc., California, USA) equipped with a 100 tray autosampler, split/splitless inlet, FID detector, and 3396 integrator and a Supelcowax10 30M column (0.32 mm ID, 0.25 Mm film thickness, Supelco Inc, Pennsylvania, USA).

Sugars, namely mannose, galactose, arabinose, glucose, and xylose, were analyzed on a Dionex HPLC system equipped with an AutoSampler AS50, Gradient Pump GP50 and Electrochemical Detector ED50A. The mobile phase was ultra-pure (distilled and de-ionized) water and was maintained at a flow rate of 1.0 mL/min for 40 minutes, followed by a 0.025M sodium hydroxide wash for 20 minutes to release the retained molecules. A post column, pre-detector, injection of 0.025M sodium hydroxide was incorporated at 1 mL/min using a TTL pump to increase the detection sensitivity. The injection volume of the sample was 25 μL and the column temperature was a constant 35° C.

Standard sugar solutions were prepared using powdered reagents all obtained from Fisher Scientific. The powders were mixed with de-ionized distilled water to create stock solutions of 5 g/L. Standards were prepared, when required, via serial dilution. Fructose was used as an internal standard at a concentration of 1.0 g/L. All stock solutions were kept at 4° C. when not in use.

Moisture Content. Pulp samples were dried overnight at 105° C. and moisture content was calculated by the difference in weight between wet and dry samples.

### EXAMPLE 4

### Comparative Analysis of Treated and Untreated OCC Samples

Treated and untreated OCC samples at 2% consistency were hydrolyzed with 20 FPU of enzymes per gram of OCC. Results are shown in FIG. 3. Approximately 20 g/L of hexose sugars is the representative of 100% conversion of the cellulose in these examples. Hexose sugars are measured in g/L and are shown to increase over the course of time in enzymatic hydrolysis.

Line A in FIG. 3 models data for hexose sugars in g/L versus time in hours of an OCC sample hydrolyzed with 20 FPU of enzymes per gram of untreated OCC. The data shows relatively low levels of conversion to sugar results, up to about 12.8 g/L (about 60% conversion after 72 hours), as compared to the treated model (line A). The Kappa value for the untreated OCC was 92.

Line B in FIG. 3 models data for hexose sugars in g/L versus time in hours of an OCC sample hydrolyzed with 20 FPU of enzymes per gram of OCC treated with oxygen and caustic for lignin deactivation processing. The oxygen/caustic lignin deactivation processing for this example included a two-stage oxygen treatment: stage 1 was run with oxygen in excess, 6% NaOH of the dried sample weight, at 105 C for 1 hour, resulting in a final pH of 12.27 and final Kappa value of 54.3; stage 2 was run with oxygen in excess, 2% NaOH of the dried sample weight, at 105 C for 1 hour, resulting in a final pH of 11.7 and final Kappa value of 40.9. The overall yield for the treated sample was 97%. Due the inclusion of lignin deactivation processing, the line B model for the conversion of starting material to sugar increases to about 19.5 g/L for the sample hydrolyzed with 20 FPU of enzymes per gram of OCC (about 97% conversion after 72 hours), a 51% increase over the results achieved with untreated OCC (line A).

### EXAMPLE 5

### Comparative Analysis of Fiber Type and Other Treatment Variables

Total sugar yield was measured as a function of fiber type and treatment variables. Three different lignocellulosic material types were used as samples: liner fibers (100% virgin softwood fibers), medium fibers (100% virgin hardwood fibers), and OCC fibers (non-virgin softwood fibers, hardwood fibers, and starch). In addition, different oxygen treatments were used on the various fiber samples, with end kappa values and total sugar yields recorded for each fiber sample. Results are shown in FIG. 5.

A 100% virgin softwood fiber sample was treated with oxygen and caustic in a lignin deactivation process at 105 C and 100 psi. Oxygen was provided to the sample in excess, and caustic was provided in amount of 5% of the dried fiber weight. The resulting Kappa number for the sample was 59 and the total sugar yield was about 83%, which is the highest sugar yield of any of the samples, showing that a relatively low amount of caustic is required for improved softwood fiber conversion to sugar.

100% virgin hardwood fiber samples were treated with oxygen and caustic in a lignin deactivation process at 105 C and 100 psi. Oxygen was provided to the samples in excess, and caustic was provided in amounts of 5%, 7.5%, and 10% of the dried fiber weight, respectively. With increasing caustic amounts, the Kappa number for the 100% virgin hardwood fiber samples was shown to decrease (92, 78.2, 71.4) and the total sugar yield was shown to increase (66, 74, 77).

Two OCC samples were treated with oxygen and caustic in a lignin deactivation process at 105 C and 100 psi. One sample was left untreated. Oxygen was provided to the treated samples in excess, and caustic was provided in amounts of 3% and 8% of the dried fiber weight, respectively. With increasing caustic amounts, the Kappa number for the OCC samples was shown to decrease (92, 72, 42.1) and the total sugar yield was shown to increase (40, 64, 79).

A summary of the experimental results are as follows:

| Lignocellulosic | | Kappa | Total Sugar |
|---|---|---|---|
| Material Type | Caustic % | Number | Yield |
| 100% virgin SW | 5% | 59 | 83 |
| 100% virgin HW | 5% | 92 | 66 |
| 100% virgin HW | 7.5% | 78.2 | 74 |
| 100% virgin HW | 10% | 71.4 | 77 |
| OCC | untreated | 92 | 40 |
| OCC | 3% | 72 | 64 |
| OCC | 8% | 42.1 | 79 |

The results show that different treatments may provide optimal results for lignin deactivation in softwood, hardwood, and mixed OCC samples. In that regard, softwood pulp samples can be processed with about 5% caustic, and hardwood pulp samples are optimally processed with an amount of caustic that will yield high amounts of sugar in hydrolysis balanced against chemical costs and carbohydrate losses occurring in the pretreatment. In one embodiment, the amount of caustic for hardwood pulp samples may be about 5-10%. In one embodiment, the amount of caustic for hardwood pulp samples may be about 5-10%. However, it is possible that lower levels of caustic may be effective for hardwood treatment, because hardwood is generally less recalcitrant overall than softwood. Therefore, by using different amounts of caustic in each portion, less total caustic to process a combined softwood and hardwood feedstock can be reduced by separating the softwood from the hardwood. This translates into lower chemical usage and a higher overall yield, because higher caustic results in more carbohydrate degradation in addition to lignin removal and deactivation.

Moreover, the results show that high sugar yields can be achieved for 100% hardwood samples, even with a Kappa value that is relatively higher than the Kappa value achieved for 100% softwood samples. This means that ultimate treatment of the hardwood fiber from recovered paper, like OCC, depends on the as-received Kappa level of the hardwood fibers, which is generally significantly higher than the as-received Kappa level of the softwood fibers because hardwood is generally processed less than softwood in the papermaking process.

### EXAMPLE 6

### Exemplary Lignocellulosic Ethanol Plant

In a lignocellulosic ethanol plant, the residence time in a conventional agitated tank in batch mode is at least 48 hours for enzymatic hydrolysis. If the plant capacity is 30 million gallons/year of ethanol and the consistency during enzymatic hydrolysis is 10%, the volume equivalent of 42 thirty-foot diameter (i.e., 17,000 ft³, 130,000 gallon) tanks are required. At 10% consistency, turbulence in the tank is essentially damped out until enzymatic hydrolysis progresses and the apparent viscosity of the stock decreases.

Adding a refiner recycle loop to the system that recirculates about 700 gallons per minute to turn over each tank about every three hours improves tank residence time significantly. Assuming a 20% enzymatic hydrolysis rate increase is obtained by the refiner loop compared to a conventional agitated tank without a refiner loop, the residence time requirement will decrease from 48 hours to 40 hours. Assuming each tank turns over twelve times, for the same output of hydrolyzed stock, the total number of tanks will decrease from 42 to 35. In addition, enzyme costs will be reduced as process efficiencies increase and process bottlenecks are reduced.

### EXAMPLE 7

### Exemplary Lignocellulosic Ethanol Plant

In a continuous process having refiners in-line between tanks with similar parameters as the batch process described in EXAMPLE 6, the capacity of the refiners will be larger than the recycle loop refiner described in the batch process, at approximately 1800 gallons per minute to allow full flow refining. This refining process will be a proportionally more intense treatment for the lignocellulosic stock than the three-hour refining loop described above in EXAMPLE 6, but will be more effective, resulting in a 40% enzymatic hydrolysis rate increase. This rate improvement will decrease the number of tanks required from 42 to 30 for the same output of hydrolyzed stock.

### EXAMPLE 8

### Comparative Analysis of Refining Conditions for OCC Samples

Using a Valley Beater lab refiner having a recycle loop, 2% consistency OCC feedstock was run at five sample conditions, Samples A-D, with total sugar conversion rates shown in FIG. 9. Experimental results of total sugar conversion versus time for OCC samples are shown in FIG. 9. The OCC samples include Samples A-D, defined as follows:

| Sample | Description |
|---|---|
| A | no refining |
| B | 2 hours of refining prior to enzymatic hydrolysis |
| C | 4 hours of refining prior to enzymatic hydrolysis |
| D | simultaneous refining and enzymatic hydrolysis |

Enzymatic hydrolysis was performed on Sample A in a shaker flask (without pre-hydrolysis or simultaneous refining of the stock) for a period of 24 hours for base comparison. The enzyme loading in the shaker flask was at 15 FPU of enzymes per gram of OCC, 50 C and 4.8 pH in a buffered solution.

The remaining OCC stock was refined in the lab refiner for 2 hours under standard, low intensity refining load (corresponding to about 0.5 kg/cm on the refiner place in the lab refiner), a temperature of 50 C, and in a buffer solution at 4.9 pH. Sample B was sampled from the refiner after 2 hours of refining, and enzymatic hydrolysis was performed on Sample B in a shaker flask for a period of 24 hours. The enzyme loading in the shaker flask was at 15 FPU of enzymes per gram of OCC.

The remaining OCC stock was refined in the lab refiner for an additional 2 hours (total 4 hours of refining) under the same conditions: under standard load, at temperature of 50 C, and in a buffer solution at 4.9 pH. Sample C was sampled from the refiner after 4 hours of refining, and enzymatic hydrolysis was performed on Sample C in a shaker flask for a period of 24 hours. The enzyme loading in the shaker flask was at 15 FPU of enzymes per gram of OCC.

The remaining OCC stock was refined in the lab refiner for an additional 20 hours (total 24 hours of refining) under minimal load (corresponding to about 0 kg/cm on the refiner place in the lab refiner) with 15 minutes on, followed by 15 minutes off for 20 hours. The refiner conditions were maintained at temperature of 50 C and in a buffer solution at 4.9 pH. After 24 hours of refining, enzymatic hydrolysis was performed on Sample C in a shaker flask for a period of 24 hours. The enzyme loading in the shaker flask was at 15 FPU of enzymes per gram of OCC.

In a separation run at the same conditions, OCC stock was subjected to refining and simultaneous enzymatic hydrolysis in the lab refiner for 24 hours. Refining was run for 4 hours under standard load, at temperature of 50 C, and in a buffer solution at 4.9 pH. After 4 hours, the refiner was run an additional 50 hours (total 24 hours of refining and enzymatic hydrolysis) under minimal load with 15 minutes on, followed by 15 minutes off. The refiner conditions were maintained at temperature of 50 C and in a buffer solution at 4.9 pH. The enzyme loading in the refiner was at 15 FPU of enzymes per gram of OCC.

The experimental results of total sugar conversion versus time for Samples A-D are shown in FIG. 9. Sample D (simultaneous refining and enzymatic hydrolysis) showed a greater increase in sugar conversion rate than the other samples, Sample A (no refining) and Samples B and C (refining prior to enzymatic hydrolysis, 2 hours and 4 hours, respectively), over time and a higher sugar conversion rate than the other samples at 24 hours. Samples B and C (refining prior to enzymatic hydrolysis) showed a greater increase in sugar conversion rate than Sample A (no refining) over time, with Sample C (4 hours refining prior to enzymatic hydrolysis) showing a greater increase over time than Sample B (2 hours refining prior to enzymatic hydrolysis).

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A system for enzymatic hydrolysis of lignocellulosic materials, comprising:
(a) a reactor vessel configured to contain a mixture of lignocellulosic stock and enzymes, the reactor vessel including a first agitator for mixing in the reactor vessel;
(b) a recycle loop coupled to the reactor vessel for recycling the mixture from and returning to the reactor vessel; and
(c) a second agitator for mixing in the recycle loop.

2. The system of claim 1, wherein the second agitator is selected from the group consisting of a refiner, a nozzle, and a static mixer.

3. The system of claim 1, wherein the recycle loop includes a pump and extends from a bottom portion of the reactor vessel to a top portion of the reactor vessel.

4. The system of claim 1, wherein the second agitator is configured to generate a higher turbulence intensity in the recycle loop than the first agitator generates in the reactor vessel.

5. The system of claim 4, wherein the higher turbulence intensity in the recycle loop promotes enzyme mass transfer without enzyme deactivation.

6. The system of claim 1, wherein the mixture of lignocellulosic stock and enzymes has a consistency in the range of about 1 to about 30 percent.

7. A system for enzymatic hydrolysis of lignocellulosic materials, comprising:
(a) a first reactor vessel configured to contain a mixture of lignocellulosic stock and enzymes, the first reactor vessel including a first agitator and a first vessel feed line; and
(b) a second agitator for promoting mixing in the first vessel feed line.

8. The system of claim 7, wherein the first vessel feed line is a recycle line.

9. The system of claim 7, wherein the system includes a second reactor vessel and wherein the first vessel feed line extends from the first reactor vessel to the second reactor vessel.

10. The system of claim 7, wherein the second agitator is configured to generate a higher turbulence intensity in the first vessel feed line than the first agitator generates in the first reactor vessel, wherein the higher turbulence intensity in the first vessel feed line promotes enzyme mass transfer without enzyme deactivation.

11. A method of enzymatic hydrolysis of lignocellulosic materials, comprising:
(a) in a reactor vessel, agitating a mixture of lignocellulosic stock and enzymes;
(b) recycling the mixture in a recycle loop extending from and returning to the reactor vessel; and
(c) further comminuting the mixture in the recycle loop for promoting effective enzyme mass transfer to and from lignocellulosic fibers in the lignocellulosic stock without enzyme deactivation.

12. The method of claim 11, wherein comminuting the mixture in the recycle loop includes refining the mixture in a refiner, agitating the mixture in a smaller vessel, or mixing the mixture with a nozzle mixing device.

13. The method of claim 11, further comprising feeding additional enzymes to the reactor vessel, wherein the enzymes are fed to the recycle loop before the mixture is agitated in the recycle loop.

14. The method of claim 11, further comprising feeding additional lignocellulosic stock to the reactor vessel, wherein the lignocellulosic stock is fed to the recycle loop before the mixture is agitated in the recycle loop.

15. The method of claim 11, further comprising feeding additional enzymes to the reactor vessel, wherein the enzymes are fed directly to the reactor vessel by a vessel feed line.

16. The method of claim 11, further comprising feeding additional lignocellulosic stock to the reactor vessel, wherein the lignocellulosic stock is fed directly to the reactor vessel by a vessel feed line.

17. A method of enzymatic hydrolysis of lignocellulosic materials, comprising:
(a) in a first reactor vessel, agitating a mixture of lignocellulosic stock and enzymes, the first reactor vessel including a first agitator and a first vessel feed line; and
(b) further agitating the mixture in the first vessel feed line for promoting effective mass transfer of the enzymes to and from lignocellulosic fibers in the lignocellulosic stock without enzyme deactivation.

18. The method of claim 17, wherein the first vessel feed line is a recycle line.

19. The method of claim 17, wherein the system includes a second reactor vessel and wherein the first vessel feed line extends from the first reactor vessel to the second reactor vessel.

20. The method of claim 17, wherein the agitation in the first vessel feed line generates a higher turbulence intensity in the first vessel feed line than the agitation in the first reactor vessel generates, wherein the higher turbulence intensity in the first vessel feed line promotes enzyme mass transfer without enzyme deactivation.
